(19) United States
(12) Patent Application Publication
(10) Pub. No.: US 2013/0012758 A1
(43) Pub. Date: Jan. 10, 2013

(54) MAGNETICALLY-CONTROLLABLE NANOMETRIC POROUS DRUG CARRIER

(76) Inventors: San-Yuan CHEN, Hsinchu City (TW); Po-Jung CHEN, Changhua County (TW); Dean-Mo LIU, Hsinchu County (TW); Shang-Hsiu HU, Taoyuan County (TW)

(21) Appl. No.: 13/286,981

(22) Filed: Nov. 1, 2011

(30) Foreign Application Priority Data
Jul. 4, 2011 (TW) 100123515

(51) Int. Cl. A61N 2/10; B82Y 5/00

(52) U.S. Cl. 600/12; 977/906

(57) ABSTRACT

The present invention discloses a magnetically-controllable nanometric porous drug carrier, wherein an organic or inorganic matrix is used to carry the drug, and wherein magnetic nanoparticles having magnetosensitivity are used to encapsulate the surface of the matrix and seal the drug inside the matrix. An external magnetic field is used to control the removal rate of the magnetic nanoparticles and control the behavior and rate of drug release.

### BACKGROUND OF THE INVENTION

1. Field of the Invention

The present invention relates to a nanometric drug carrier, particularly to a magnetically-controllable nanometric porous drug carrier.

2. Description of the Related Art

So far, there have been many studies related to nanometric porous silica structure, wherein drug molecules are contained inside the pores of silica to form a nanometric porous silica drug carrier. However, some problems still exist therein. For example, as the nanometric porous silica drug carrier has open pores, drug is likely to leak during transportation. Although iron oxide nanoparticles (normally used as the contrast agent of MR (Magnetic Resonance) imaging but usually fail to work well) can be implanted into or carried by the existing nanometric porous drug carrier, they cannot function to control drug release. Although the drug molecules inside the pores can be released via some mechanism, such as diffusion, the timing and dose of drug release is hard to be controlled appropriately.

Accordingly, the present invention proposes a novel magnetically-controllable nanometric porous drug carrier to overcome the abovementioned problems.

### SUMMARY OF THE INVENTION

The primary objective of the present invention is to provide a magnetically-controllable nanometric porous drug carrier, wherein magnetic iron oxide particles perfectly cap the drug carrier to reduce drug leakage.

Another objective of the present invention is to provide a magnetically-controllable nanometric porous drug carrier, wherein an external magnetic field is used to control drug release, whereby the drug is released to the target nidus precisely, and whereby is reduced the drug dose and decreased the harm to the human body.

Yet another objective of the present invention is to provide a magnetically-controllable nanometric porous drug carrier, wherein the intensity of an external magnetically field is varied to control the removal of the magnetic nanoparticles, whereby is controlled the behavior and rate of drug release.

A further objective of the present invention is to provide a magnetically-controllable nanometric porous drug carrier, wherein the magnetic nanoparticles are used to monitor the position of the drug carrier, the tumor or the sick tissue.

To achieve the abovementioned objectives, the present invention proposes a magnetically-controllable nanometric porous drug carrier, which comprises a matrix having several pores; at least one drug contained inside the pores; and at least one removable cap sealing the pores and containing several magnetic nanoparticles that can be removed by an external magnetic field.

The present invention also proposes a magnetically-controllable nanometric porous drug carrier, which comprises a matrix made of a two-phase organic material; at least one lipophilic drug microemulsified together with the matrix and then wrapped in the matrix; at least one removable cap sealing the matrix for storing the drug inside the matrix and containing several magnetic nanoparticles that can be removed by an external magnetic field.

Below, the embodiments are described in detail to make easily understood the objectives, technical contents, characteristics and accomplishments of the present invention.

### BRIEF DESCRIPTION OF THE DRAWINGS

FIG. 1(a) schematically shows a magnetically-controllable nanometric porous drug carrier according to a first embodiment of the present invention;

FIG. 1(b) schematically shows that an external magnetic field enables the drug release of the magnetically-controllable nanometric porous drug carrier according to the first embodiment of the present invention;

FIG. 2 shows TEM images of the present invention wherein (a) shows the porous silica nanoparticles fabricated according to the first embodiment, (b) shows the iron oxide nanoparticles used in the first embodiment, and (c) shows the nanometric porous drug carrier fabricated according to the first embodiment;

FIGS. 3(a)-3(c) respectively show the result of small-angle X-ray diffraction, the result of large-angle X-ray diffraction, and the magnetic hysteresis curve obtained with SQUID, for the magnetically-controllable nanometric porous drug carrier according to the first embodiment of the present invention;

FIG. 4 shows the drug-releasing state after an external magnetic field is respectively applied for 0, 1, 3, and 5 minutes to the magnetically-controllable nanometric porous drug carrier according to the first embodiment of the present invention;

FIG. 5 shows TEM images of the present invention wherein (a)-(c) respectively show of the nanometric porous drug carrier stimulated by an external magnetic field for 1, 3, and 5 minutes according to the first embodiment;

FIG. 6 schematically shows the process of using an organic material as the matrix to fabricate a magnetically-controllable nanometric porous drug carrier according to a second embodiment of the present invention;

FIG. 7 respectively shows TEM images and the size distributions according to the second embodiment of the present invention wherein (a) and (b) respectively show TEM images of the iron oxide nanoparticles and the nanometric porous drug carrier, and (c) and (d) respectively show the size distributions of the iron oxide nanoparticles and the nanometric porous drug carrier;

FIG. 8 shows the results of the large-angle X-ray diffraction test of the nanometric porous drug carrier according to the second embodiment of the present invention; and

FIG. 9 shows the drug-releasing state after an external magnetic field is respectively applied for 0, 1, 2, and 4 minutes to the magnetically-controllable nanometric porous drug carrier according to the second embodiment of the present invention.

### DETAILED DESCRIPTION OF THE INVENTION

The spirit of the present invention is to combine materials and drugs to form a multifunctional nanometric drug carrier, which can release the drugs to the target cancered tissue and implement drug release surveillance.

The magnetically-controllable nanometric porous drug carrier of the present invention adopts an organic or inorganic matrix to carry the drug and uses magnetic nanoparticles having magnetosensitivity to cap the matrix and seal the drug. Further, the magnetic nanoparticles may even form a nanocapsule to completely wrap the drug.

When no external magnetic field is applied, almost none drug is released from the nanocapsule. Thus is greatly reduced the side effect of medicine, such as a high-toxicity anticancer drug. When the nanocapsule reaches the target, such as a tumor, the magnetic iron oxide nanoparticles can function as the contrast agent of MR imaging to detect the position of the tumor. Further, an external magnetic field can control the drug to release locally around the tumor to attain the optimal therapeutic effect.

The matrix is made of porous silica or a bipolar polymer. The bipolar polymer is selected from a group consisting of polyvinyl alcohol (PVA), polystyrene sulphonate (PSS), Poly(allylamine hydrochloride) (PAH), and Polyvinylpyrrolidone (PVP). The magnetic nanoparticles are made of a material selected from a group consisting of ferric oxide (Fe₂O₃), ferric ferrous oxide (Fe₃O₄), cobalt iron oxide (CoFe₂O₄), manganese iron oxide (MnFe₂O₄) and gadolinium oxide (Gd₂O₃).

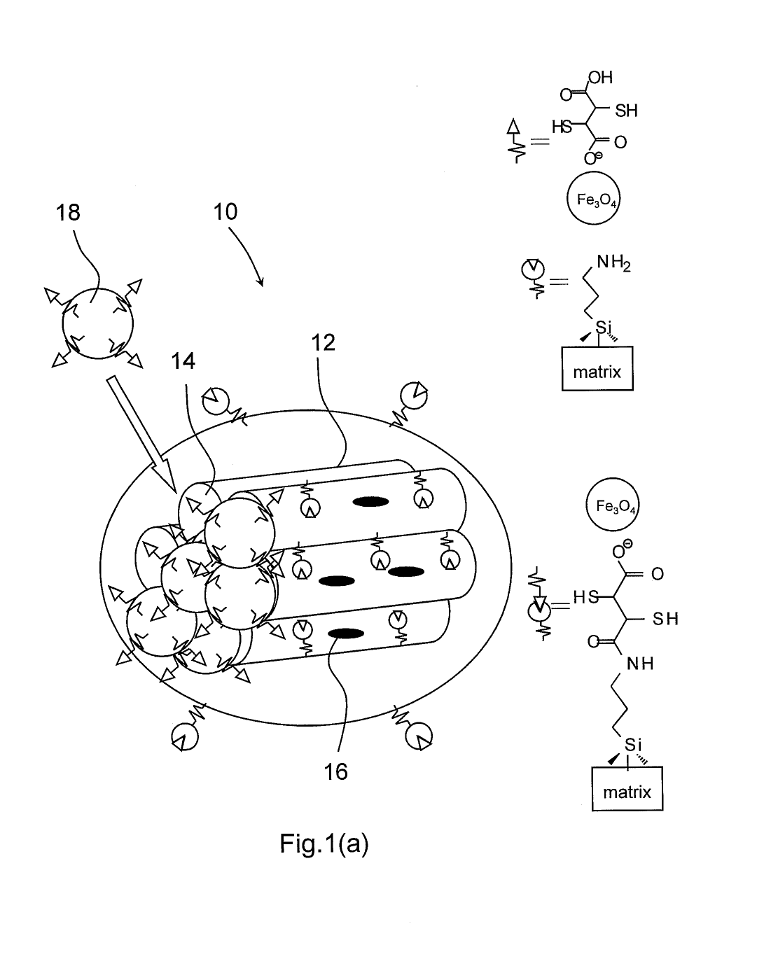

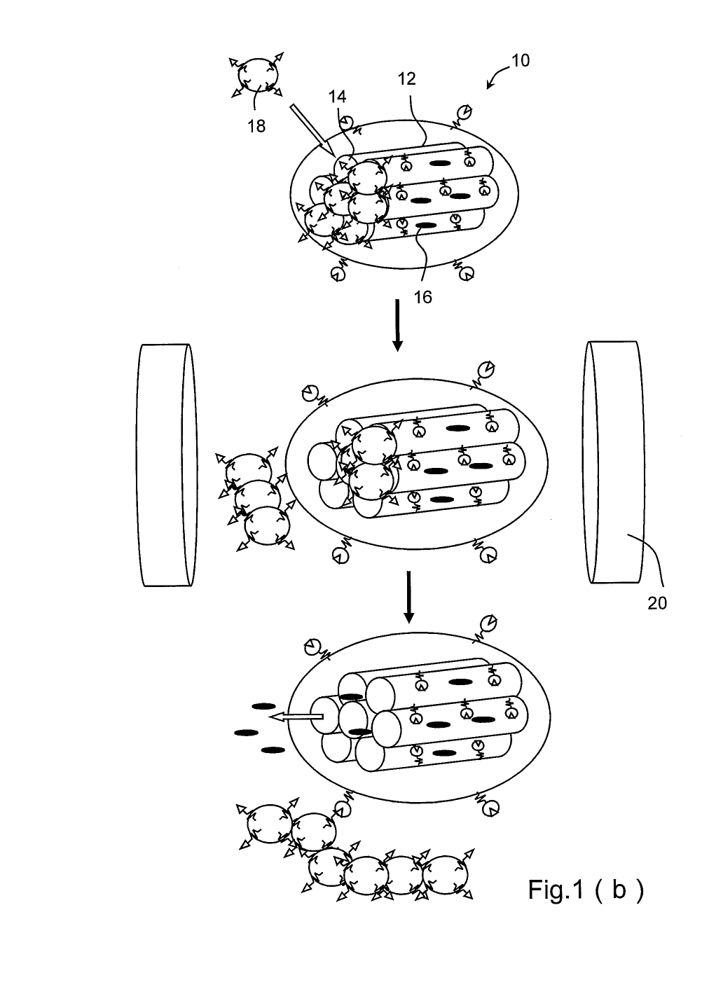

Refer to FIG. 1(a) and FIG. 1(b). FIG. 1(a) is a diagram schematically showing a magnetically-controllable nanometric porous drug carrier according to a first embodiment of the present invention. FIG. 1(b) is a diagram schematically showing an external magnetic field controls the drug release of the magnetically-controllable nanometric porous drug carrier according to the first embodiment of the present invention. The magnetically-controllable nanometric porous drug carrier 10 of the present invention comprises a matrix 12 (a silica nanoparticle in the first embodiment) having several pores 14; at least one drug 16 ((S)-(+)-camptothecin (CPT) in the first embodiment) filled into the pores 14; and at least one removable cap 18 sealing the pores 14, containing several magnetic nanoparticles, and controlled by an external magnetic field 20 to release the drug 16.

As shown in the drawings, a chemical bonding may form between the iron oxide nanoparticle and the silica nanoparticle, enabling the iron oxide nanoparticle to adhere to the pore of the silica nanoparticle. In the first embodiment, an amino group (NH₃) of the silica nanoparticle is covalently bonded to a carboxylic acid group (COOH) group of the iron oxide nanoparticle to form the chemical bonding.

Below is described in detail the process for fabricating the magnetically-controllable nanometric porous drug carrier according to the first embodiment of the present invention. Firstly, place CTAB (Cetyltrimethylammonium bromide) in water and heat them to a temperature of 80° C. to obtain a uniform solution. Next, add TEOS (Tetraethylorthosilicate), APTMS (3-aminopropyltrimethoxy silane) and NaOH (Sodium hydroxide) into the solution to form a mixture solution, and agitate the mixture solution for 2 hours to activate the reaction thereof. Next, centrifugally collect white precipitation from the mixture solution, and clean the precipitation several times with a solution of methanol and water by a ratio-of 1:1. Next, collect the precipitation and dissolve it in methanol. Next, add hydrochloric acid (HCl) into the mixture solution of precipitation and methanol, and agitate the mixture solution for 24 hours to activate the reaction thereof. Then, collect the precipitation, which is the porous silica nanoparticles with the surface thereof modified to have amino groups.

In the first embodiment, the anticancer drug is exemplified by (S)-(+)-camptothecin (CPT). The anticancer drug and the porous silica nanoparticles are dissolved in DMSO (Dimethyl sulfoxide). Then, DMSO is removed by evacuation. Thus, the drug is sucked into the pores of the porous silica nanoparticles.

Next, add DMSA-modified iron oxide nanoparticles and a cross-liking agent EDC to the drug-containing silica nanoparticles, and dissolve them in deionized water, and agitate them for 24 hours to activate the reaction thereof, wherein DMSA denotes meso-2,3-dimercaptosuccinic acid and EDC denotes 1-Ethyl-3-(3-dimethylaminopropyl)carbodiimide. Thus is obtained a nanometric porous carrier covered by removable nanometric caps, as shown in FIG. 1(a).

The surface of the DMSA-modified iron oxide nanoparticles has thiol (HS) groups. The abovementioned process is undertaken at an ambient temperature and would not damage the activity of the drug.

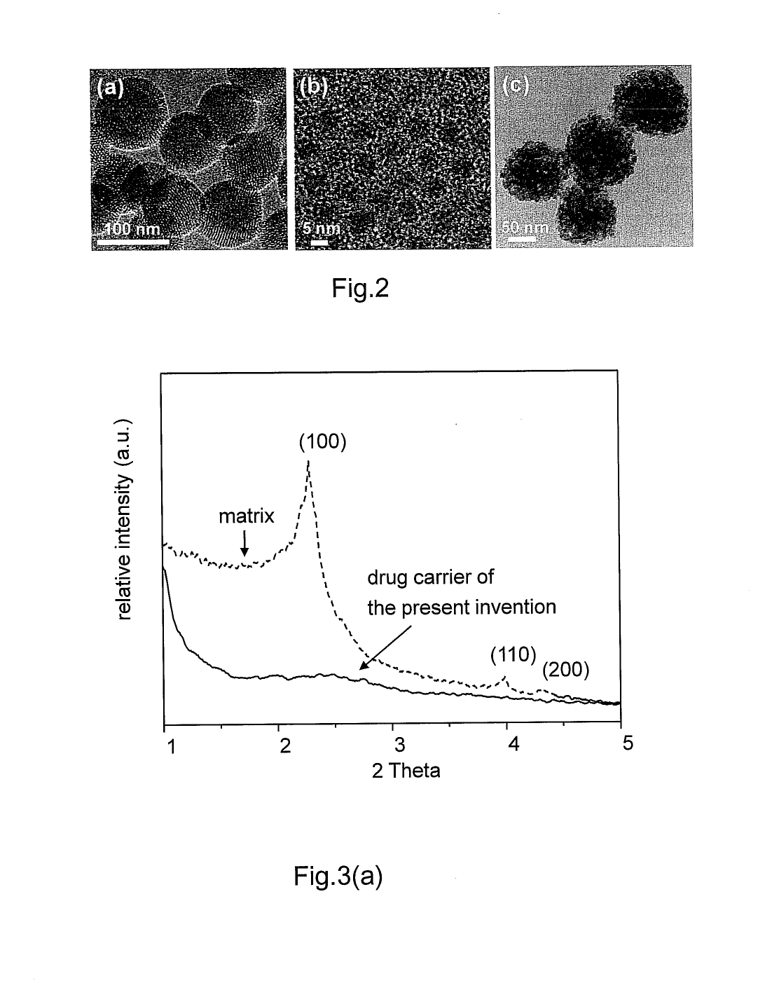

Refer to FIG. 2. Fig.(a) shows a TEM (Transmission Electron Microscope) image of the porous silica nanoparticles fabricated according to the present invention. Fig.(b) shows a TEM (Transmission Electron Microscope) image of the iron oxide nanoparticles used in the present invention. Fig.(c) shows a TEM (Transmission Electron Microscope) image of the nanometric porous drug carrier fabricated according to the present invention. The abovementioned images prove that the iron oxide nanoparticles successfully cap the nanometric silica carrier.

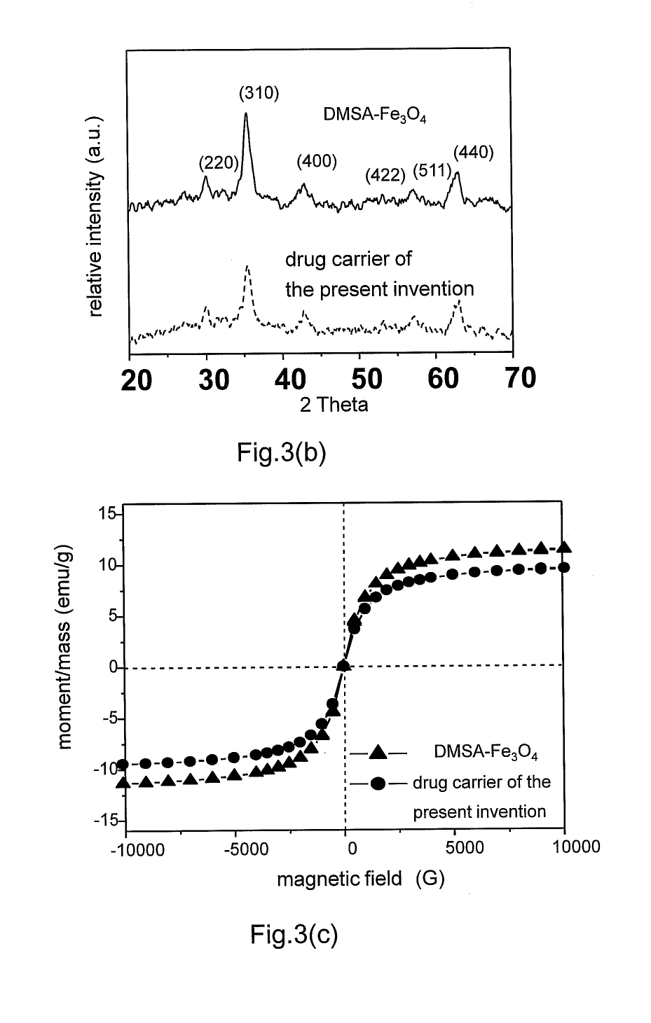

Refer to FIGS. 3(a)-3(c). The crystalline structures are identified with XRD (X-ray diffraction) in small angles and large angles. In the cases of small-angle diffraction, the porous silica nanoparticles have absorption peaks at double angles of 2.3, 3.9 and 4.3 degrees; the porous silica drug carriers capped by removable iron oxide nanoparticles have none absorption peak at double angles of 2.3, 3.9 and 4.3 degrees. The small-angle diffraction proves that the iron oxide nanoparticles completely wrap the surface of the porous silica drug carrier. However, the absorption peaks of the iron oxide nanoparticles appear in the large-angle diffraction. The magnetosensitivity of the present invention is verified with SQUID (superconducting quantum interference device). FIG. 3(c) shows that the nanometric porous drug carrier of the present invention has superior superparamagnetism and magnetosensitivity.

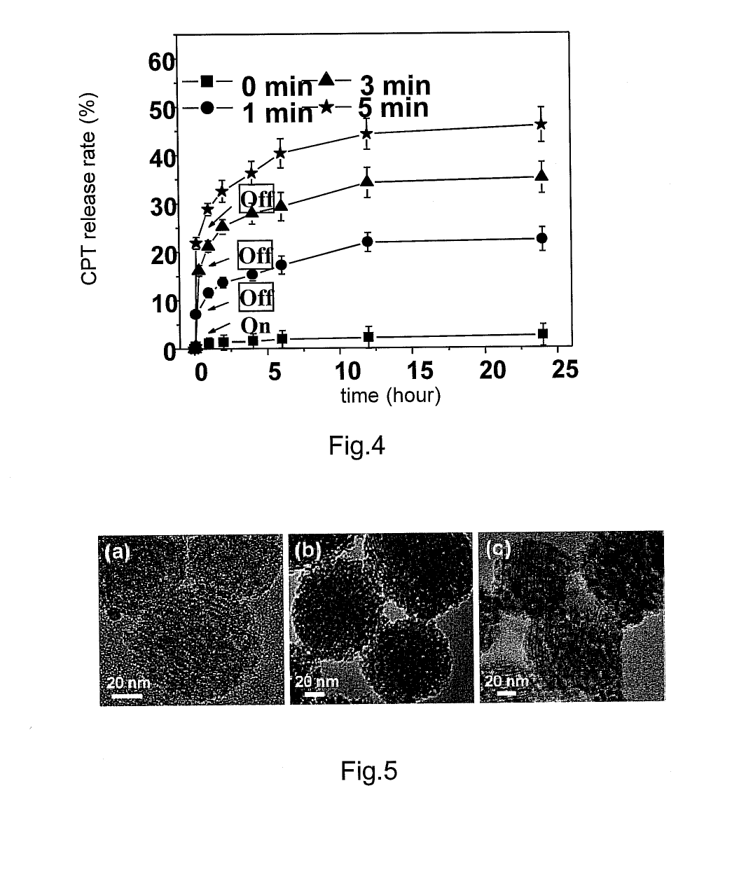

In the drug-releasing performance test, the anticancer drug (S)-(+)-camptothecin (CPT) is filled into the silica drug carriers of the present invention firstly. The drug molecules that are not wrapped inside the drug carriers are removed with a flushing method. Thus, the drug is perfectly wrapped inside the carrier and hard to leak. (S)-(+)-camptothecin (CPT) has a maximum absorption peak at a wavelength of 366 nm in UV-vis (ultraviolet visible spectroscopy) test. Such a feature is used to detect the concentration of the released drug. Refer to FIG. 4. When no external magnetic field is applied, the drug carrier of the present invention does not release any drug molecules and thus can be regarded as a long-term drug-wrapping system. After an external magnetic field has been applied to the drug carrier, there is an obvious absorption peak appearing at a wavelength of 366 nm—the absorption peak of the drug molecules. It means that the drug carrier of the present invention can react fast to release the drug after a short period of magnetic stimulus. After the external magnetic field is shut off, the drug carrier of the present invention still continues releasing the drug until equilibrium is reached. After a magnetic stimulus, a portion of iron oxide nanoparticles are detached from the surface of the nanometric porous drug carrier. Thus, a portion of the surface of the matrix is exposed, and the drug molecules are released from the pores. The undetached iron oxide nanoparticles keep on capping the surface of the matrix, preventing the rest of drug molecules from being released.

When the duration of applying an external magnetic field is increased from 1 minute to 3 and 5 minutes, the drug-releasing quantity is also increased discretely. In other words, the duration of applying an external magnetic field can be varied to manipulate the drug-releasing quantity.

In order to understand the relationship between the drug-releasing quantity and the quantity of the iron oxide nanoparticles detached from the surface of the drug carrier, we measure the magnetic stimulus duration, the quantity of the lost iron oxide nanoparticles, the quantity of the iron oxide nanoparticle attached on the surface of a single drug carrier, and the exposed area of a single drug carrier (the area not occupied by iron oxide nanoparticles), as shown in (a)-(c) of FIG. 5. Thus is obtained the relationship of the magnetic stimulus duration, the exposed area of the drug carrier, and the drug-releasing quantity. Thereby, we can control drug-releasing quantity via controlling the magnetic stimulus duration.

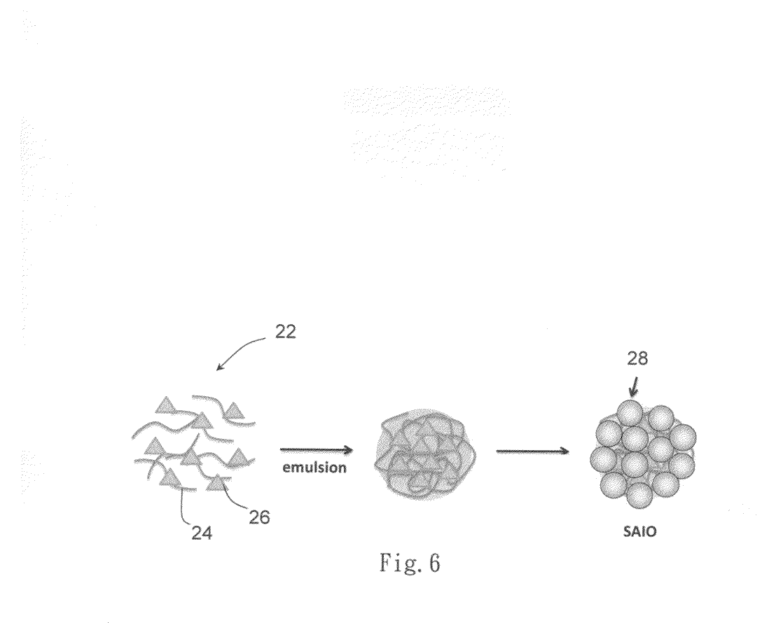

Refer to FIG. 6 a diagram schematically showing the process of using an organic material as the matrix to fabricate a magnetically-controllable nanometric porous drug carrier according to a second embodiment of the present invention. In the second embodiment, the magnetically-controllable nanometric porous drug carrier 22 (designated by SAIO in the drawing) of the present invention comprises a matrix 24 made of a polymer having a hydrophilic terminal and a lipophilic terminal (such as PVA); a lipophilic drug 26 (such as ibuprofen (IBU)) attached to the lipophilic terminal of PVA, microemulsified and then encapsulated inside the PVA matrix 24; and at least one iron oxide nanoparticle 28 electrically attached to the surface of the PVA matrix 24 to form a cap, or even a capsule, capping the PVA matrix 24. The iron oxide nanoparticles 28 can be removed magnetically to control drug release.

Below is described in detail the process for fabricating the magnetically-controllable nanometric porous drug carrier according to the second embodiment of the present invention. Firstly, dissolve PVA in water to obtain a 2 wt % solution thereof. Next, dissolve a lipophilic drug in 2 ml of chloroform (CHCl₃). Next, mix 5 ml of 2 wt % PVA solution and 2 ml of the chloroform solution of the drug uniformly, and emulsify the mixture ultrasonically for 2 minutes. The solution thus becomes light brown gradually. Next, the solution is heated to a temperature of 60° C. to evaporate the residual organic solvent (chloroform). Next, flush the product with deionized water several times. Next, add iron oxide nanoparticles to the product. Then, the iron oxide nanoparticles are attached to the surface of the PVA material to form a PVA-based and iron oxide nanoparticle-capped nanometric drug carrier.

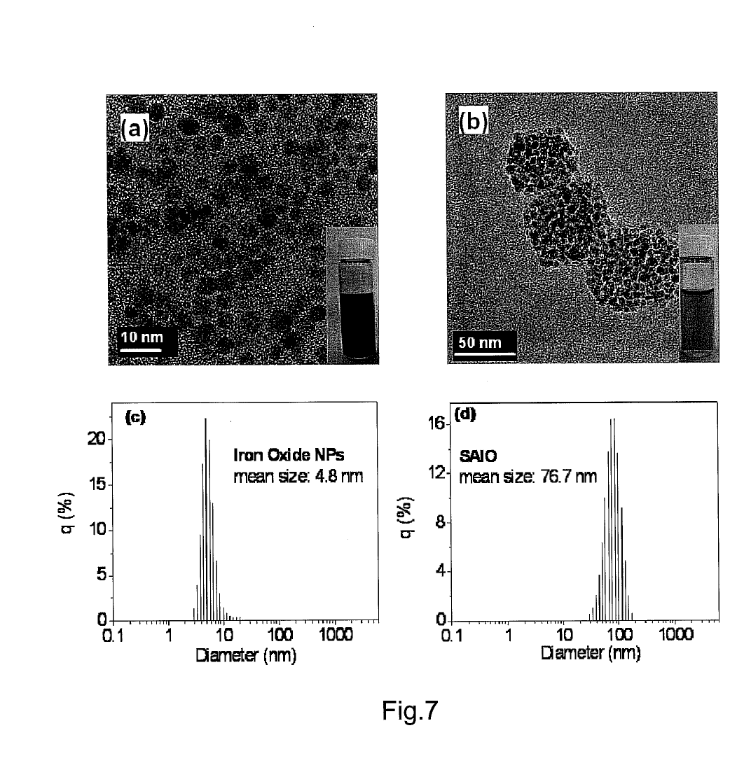

Refer to FIG. 7. As shown in (a)-(d), the TEM images and the crystalline structure analysis prove that the iron oxide nanoparticles indeed cap the PVA matrix.

In the crystalline structure analysis, a diameter analyzer is used to estimate the diameter of the iron oxide nanoparticles and the diameter of the nanometric porous drug carriers of the present invention. As shown in Fig.(c) and Fig.(d), the iron oxide nanoparticles have a diameter of about 4.8 nm, and the porous drug carriers of the present invention have a diameter of about 76.7 nm. It is found in the drawings that the diameters distribute in a pretty narrow range. It means that the nanoparticles fabricated according to the present invention have consistent sizes.

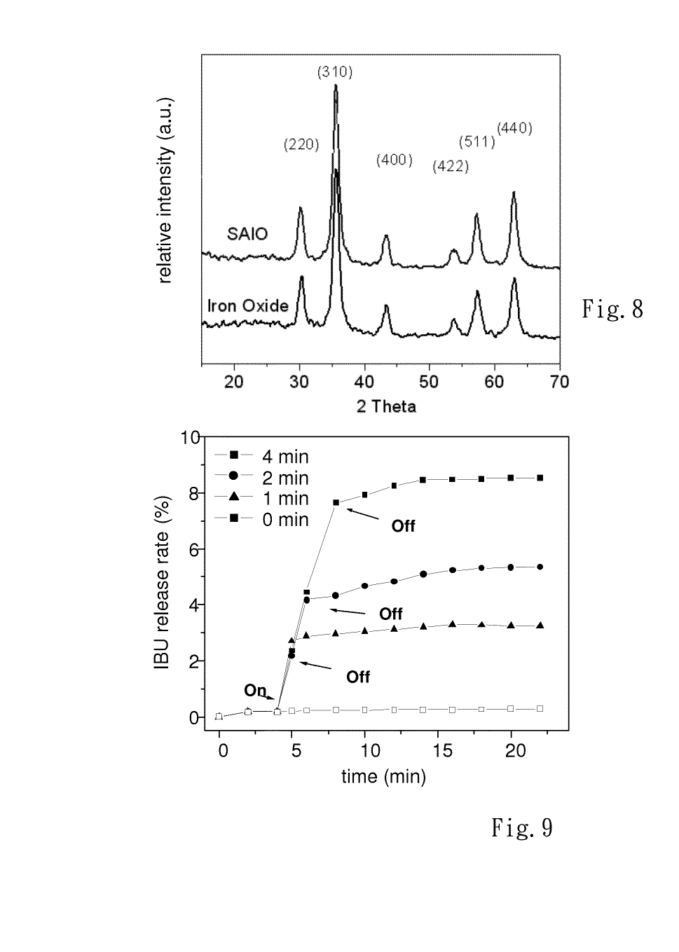

Refer to FIG. 8. Large-angle X-ray diffraction is used to examine the crystalline structures of the iron oxide nanoparticles and the porous drug carriers. It is found that the absorption peaks of the porous drug carriers are the same as the absorption peaks of the iron oxide nanoparticles. Such a result proves that the matrix is indeed completely wrapped by the iron oxide nanoparticles.

In the drug-releasing performance test, ibuprofen (IBU) is encapsulated in the porous drug carriers of the present invention firstly. The drug molecules that are not wrapped inside the drug carriers are removed with a flushing method to guarantee correctness of the drug-releasing performance test. Ibuprofen (IBU) has a maximum absorption peak at a wavelength of 264 nm in UV-vis test. Such a feature is used to detect the concentration of the released drug. Refer to FIG. 9. When no external magnetic field is applied to the nanometric porous drug carrier having a PVA-based matrix, the drug is encapsulated inside a capsule made of iron oxide nanoparticles. There may be a very small amount of drug leakage caused by too great a concentration difference therebetween. However, the amount of drug exposed outside is neglectable.

In order to understand the efficacy of the cap formed by the iron oxide nanoparticles, we compare the quantity of the drug released by the PVA-based drug carrier without the iron oxide cap with the quantity of the drug released by the PVA-based iron oxide-capped drug carrier. We found that the PVA-based drug carrier without the iron oxide cap persistently releases the drug. Such a result proves that the iron oxide capping layer can indeed prevent the drug from being released.

After an external magnetic field has been applied to the drug carrier of the present invention for one minute, there is an obvious absorption peak of the drug molecules appearing at a wavelength of 264 nm. Such a result indicates that the external magnetic field induces the iron oxide nanoparticles to separate from the PVA-based drug carrier and causes the drug molecules to be released fast via the pores of the matrix. When the duration of the magnetic stimulus is increased from one minute to 2 and 4 minutes, the quantity of released IBU is also increased discretely corresponding to the duration. In other words, the duration of applying an external magnetic field can be varied to manipulate the drug-releasing quantity. After the external magnetic field is shut off, the drug carrier of the present invention still continues releasing the drug until equilibrium is reached. This is because the magnetic field has varied the structure of the PVA-based drug carrier.

The embodiments described above are only to exemplify the present invention but not to limit the scope of the present invention. Any equivalent modification or variation according to the spirit of the present invention is to be also included within the scope of the present invention.

What is claimed is:

1. A magnetically-controllable nanometric porous drug carrier comprising
a matrix having several pores;
at least one drug contained inside said pores; and
at least one removable cap sealing said pores to store said drug inside said pores and containing several magnetic nanoparticles that can be removed by an external magnetic field.

2. The magnetically-controllable nanometric porous drug carrier according to claim 1, wherein said matrix is made of silica, and wherein said magnetic nanoparticles are made of iron oxide, and wherein said magnetic nanoparticles is joined to said matrix via chemical bonding.

3. The magnetically-controllable nanometric porous drug carrier according to claim 1, wherein an external magnetic field is used to manipulate said cap and control said drug to be released continuously, discretely or abruptly.

4. The magnetically-controllable nanometric porous drug carrier according to claim 1, wherein said matrix is made of silica and has a size of 20-5000 nm, and wherein said pores has a size of 1-30 nm, and wherein said magnetic nanoparticles are made of ferric ferrous oxide (Fe₃O₄) and has a size of 5-50 nm.

5. The magnetically-controllable nanometric porous drug carrier according to claim 1, wherein said magnetic nanoparticles are made of a material selected from a group consisting of ferric oxide (Fe₂O₃), ferric ferrous oxide (Fe₃O₄), cobalt iron oxide (CoFe₂O₄), manganese iron oxide (MnFe₂O₄) and gadolinium oxide (Gd₂O₃).

6. A magnetically-controllable nanometric porous drug carrier comprising
a matrix made of a two-phase polymeric material;
at least one lipophilic drug microemulsified together with said matrix and then encapsulated inside said matrix; and
at least one removable cap sealing said matrix to store said drug inside said matrix and containing several magnetic nanoparticles that can be removed by an external magnetic field.

7. The magnetically-controllable nanometric porous drug carrier according to claim 6, wherein said matrix is made of a material selected from a group consisting of polyvinyl alcohol (PVA), polystyrene sulphonate (PSS), Poly(allylamine hydrochloride) (PAH), and Polyvinylpyrrolidone (PVP), and wherein said magnetic nanoparticles are made of a material selected from a group consisting of ferric oxide (Fe₂O₃), ferric ferrous oxide (Fe₃O₄), cobalt iron oxide (CoFe₂O₄), manganese iron oxide (MnFe₂O₄) and gadolinium oxide (Gd₇O₃), and wherein said matrix and said magnetic nanoparticles are bridged by attraction of opposite charges.

8. The magnetically-controllable nanometric porous drug carrier according to claim 6, wherein an external magnetic field is used to manipulate said cap and control said drug to be released continuously, discretely or abruptly.

9. The magnetically-controllable nanometric porous drug carrier according to claim 6, wherein said matrix is made of PVA and has a size of 10-5000 nm, and wherein said magnetic nanoparticles are made of ferric ferrous oxide (Fe₃O₄) and has a size of 5-50 nm.
